(19) United States
(12) Patent Application Publication
(10) Pub. No.: US 2004/0229880 A1
(43) Pub. Date: Nov. 18, 2004

(54) 4-[(2,4-dichloro-5-methoxyphenyl)amino]-6-alkoxy-3-quinolinecarbonitriles for the treatment of ischemic injury

(75) Inventors: Diane Harris Boschelli, New City, NY (US); Margaret Maria Zaleska, Narberth, PA (US); Frank Charles Boschelli, New City, NY (US); Kim Timothy Arndt, Towaco, NJ (US)

Correspondence Address: WYETH, PATENT LAW GROUP, 5 GIRALDA FARMS, MADISON, NJ, 07940, US

(73) Assignee: Wyeth, Madison, NJ

(21) Appl. No.: 10/780,973

(22) Filed: Feb. 18, 2004

Related U.S. Application Data
(60) Provisional application No. 60/449,316, filed on Feb. 21, 2003.

(51) Int. Cl. A61K 31/496; A61K 31/4709

(52) U.S. Cl. 514/253.06; 514/313

(57) ABSTRACT

Compounds of the formula:

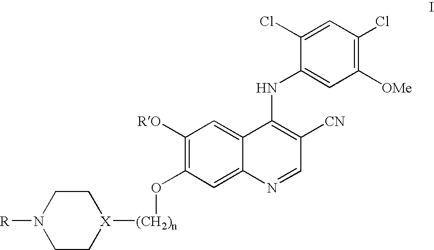

wherein:

X is N, CH

n is an integer from 1-3; and

R′ and R are independently, alkyl of 1 to 3 carbon atoms, and pharmaceutically acceptable salts thereof, with the proviso that when n is 1, X is not N; are useful for inhibiting vascular permeability caused by disease, injury, or other trauma.

This application claims priority from co-pending provisional application serial No. 60/449,316, filed on Feb. 21, 2003, the entire disclosure of which is hereby incorporated by reference.

### BACKGROUND OF THE INVENTION

Stroke is the third leading cause of death and the major cause of disability in the US, where approximately 750,000 strokes occur each year. Ischemic stroke comprises about 80% of this number, with primary intracerebral hemorrhagic stroke about 15-20%. To date, the only approved efficacious treatment for acute ischemic cerebral infarction is thrombolytic therapy by means of intravenous administration of t-PA, recombinant tissue plasminogen activator. The usefulness of this therapy is extremely limited. It must be given within a three hour window after the onset of symptoms, while a majority of patients seek and/or receive treatment after a substantial delay. In addition, treatment with t-PA carries an increased risk of causing intracerebral hemorrhage, a potentially devastating complication. Presence of hemorrhage must be ruled out prior to treatment and blood pressure must be carefully managed and monitored during and after treatment with t-PA. Currently, no neuroprotective therapy is available for treatment of ischemic stroke, hemorrhagic stroke or brain trauma. New treatments for stroke and other conditions associated with vascular permeability are greatly needed.

### DESCRIPTION OF THE INVENTION

In accordance with the present invention are provided compounds of the structural formula:

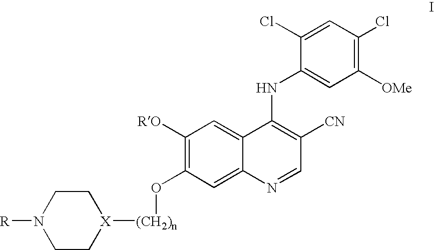

wherein:

X is N, CH

n is an integer from 1-3; and

R′ and R are independently, alkyl of 1 to 3 carbon atoms, and pharmaceutically acceptable salts thereof, with the proviso that when n is 1, X is not N.

In some preferred embodiments of the invention, R′ is methyl.

In other preferred embodiments of the invention, R is methyl or ethyl.

In still other embodiments of the invention, n is 2 or 3.

X is preferably N in some preferred embodiments of the invention.

In yet other preferred embodiments X is CH.

Pharmaceutically acceptable salts are those derived from such organic and inorganic acids as: acetic, lactic, carboxylic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

Specific compounds of the invention include:

4-[(2,4-Dichloro-5-methoxyphenyl)amino]-6-methoxy-7-[3-(4-methyl-1-piperazinyl)propoxy]-3-quinolinecarbonitrile;

4-[(2,4-Dichloro-5-methoxyphenyl)amino]-7-[3-(4-ethyl-1-piperazinyl)propoxy]-6-methoxy-3-quinolinecarbonitrile;

4-[(2,4-Dichloro-5-methoxyphenyl)amino]-6-methoxy-7-[2-(4-methyl-1-piperazinyl)ethoxy]-3-quinolinecarbonitrile;

4-[(2,4-Dichloro-5-methoxyphenyl)amino]-7-[2-(4-ethyl-1-piperazinyl)ethoxy]-6-methoxy-3-quinolinecarbonitrile;

4-[(2,4-Dichloro-5-methoxyphenyl)amino]-6-methoxy-7-[(1-methylpiperidin-4-yl)methoxy]-3-quinolinecarbonitrile;

4-[(2,4-Dichloro-5-methoxyphenyl)amino]-6-methoxy-7-[2-(1-methylpiperidin-4-yl)ethoxy]-3-quinolinecarbonitrile;

4-[(2,4-Dichloro-5-methoxyphenyl)amino]-6-methoxy-7-[3-(1-methylpiperidin-4-yl)propoxy]quinoline-3-carbonitrile;

4-[(2,4-Dichloro-5-methoxyphenyl)amino]-7-[( 1-ethylpiperidin-4-yl)methoxy]-6-methoxyquinoline-3-carbonitrile;

4-[(2,4-Dichloro-5-methoxyphenyl)amino]-6-ethoxy-7-[3-(4-methylpiperazin-1-yl)propoxy]quinoline-3-carbonitrile;

4-[(2,4-Dichloro-5-methoxyphenyl)amino]-6-ethoxy-7-[(1-methylpiperidin-4-yl)methoxy]quinoline-3-carbonitrile;

4-[(2,4-Dichloro-5-methoxyphenyl)amino]-6-ethoxy-7-[3-(4-ethylpiperazin-1-yl)propoxy]quinoline-3-carbonitrile;

4-[(2,4-Dichloro-5-methoxyphenyl)amino]-6-ethoxy-7-[3-(1-methylpiperidin-4-yl)propoxy]quinoline-3-carbonitrile;

4-[(2,4-Dichloro-5-methoxyphenyl)amino]-6-ethoxy-7-[2-(4-methyl-1-piperazinyl)ethoxy]quinoline-3-carbonitrile;

4-[(2,4-Dichloro-5-methoxyphenyl)amino]-6-ethoxy-7-[2-(1-methylpiperidin-4-yl)ethoxy]quinoline-3-carbonitrile; and

4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

The compounds of the invention are prepared as illustrated below. The compounds of this invention were prepared from: (a) commercially available starting materials (b) known starting materials which can be prepared as described in literature procedures or (c) new intermediates described in the schemes and experimental procedures herein.

Reactions are performed in a solvent appropriate to the reagents and materials employed and suitable for the transformation being effected. It is understood by those skilled in the art of organic synthesis that the various functionalities present on the molecule must be consistent with the chemical transformations proposed. When not specified, order of synthetic steps, choice of protecting groups and deprotection conditions will be readily apparent to those skilled in the art. In addition, in some instances, substituents on the starting materials may be incompatible with certain reaction conditions. Restrictions pertinent to given substituents will be apparent to one skilled in the art. Reactions were run under inert atmospheres where appropriate. Compounds of Formula I were prepared as described in Scheme 1. Compounds of Formula I wherein R′ is Me, X is N and n is 3 are readily obtained by treatment of 7-(3-chloropropoxy)-4-[(2,4-dichloro-5-methoxyphenyl)amino]-6-methoxy-3-quinolinecarbonitrile, 1, with N-alkylpiperazine such as N-methylpiperazine, N-ethylpiperazine, or N-propylpiperazine in the presence of sodium iodide either neat or in a solvent such as ethylene glycol dimethyl ether. The preparation of these compounds has been reported in the literature, [Boschelli, D. H., et. al., J. Med. Chem., 44, 3965 (2001)].

Analogously compounds of Formula I wherein R′ is Me, X is N and n is 2 are readily obtained by treatment of 7-(2-chloroethoxy)-4-[(2,4-dichloro-5-methoxyphenyl)amino]-6-methoxy-3-quinolinecarbonitrile, 2, with N-methyl or N-ethylpiperazine in the presence of sodium iodide either neat or in a solvent such as ethylene glycol dimethyl ether. The preparation of these compounds has been reported in the literature, [Ye, F. et. al., 221th National Meeting of the American Chemical Society, San Diego, Calif. (April, 2001)].

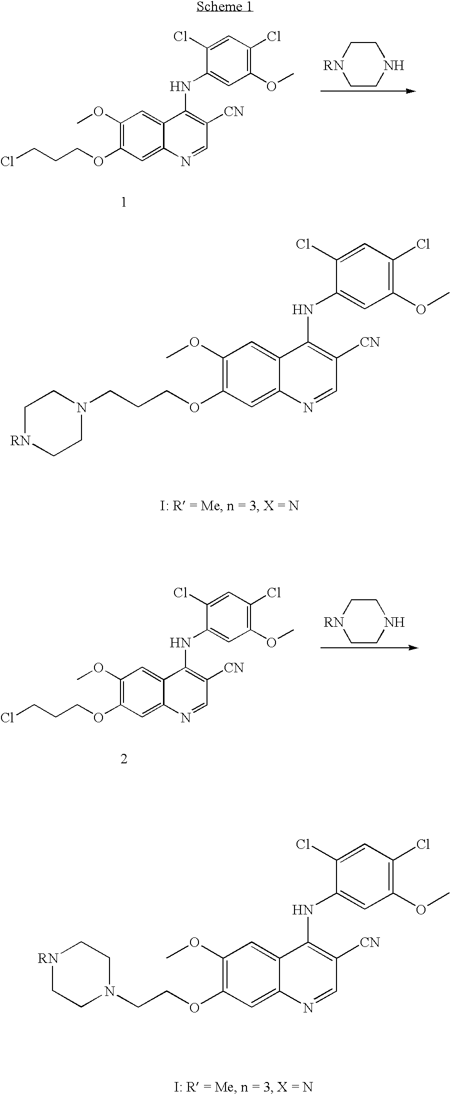

Alternatively compounds of Formula I can be prepared via a 7-fluoro-3-quinolinecarbonitrile intermediate. Preparation of this key intermediate is shown in Scheme 2. Anilines of formula 3 can be reacted with diethyl (ethoxymethylene)malonate either neat or in the presence of a cosolvent such as toluene, at temperatures ranging from 60 to 120° C. Subsequent thermal cyclization, preferably in a solvent system such as 3:1 mixture of diphenyl ether and biphenyl at elevated temperature, such as 260° C., provides compounds of formula 4. Hydrolysis of the ester group under preferably basic conditions, such as sodium hydroxide in an alcoholic solvent such as ethanol, at elevated temperatures results in compounds of formula 5. Conversion of the acid group to the primary amide can be accomplished by treatment with an activating agent such as 1,1-carbonyldiimidazole followed by the addition of either ammonia gas or preferably an aqueous solution of ammonium hydroxide. Dehydration of the primary amide group of compounds of formula 6 with a reagent such as cyanuric chloride in a solvent such as N,N-dimethylformamide provides compounds of formula 7. Alternatively, anilines of formula 3 can be treated with ethyl (ethoxymethylene)cyanoacetate either neat or in the presence of a cosolvent such as toluene, at temperatures ranging from 60 to 120° C. Subsequent thermal cyclization, preferably in a solvent system such as 3:1 mixture of diphenyl ether and biphenyl at elevated temperature, such as 260° C., provides compounds of formula 7. Reaction of 7 with a chlorinating agent such as phosphorous oxychloride gives compounds of formula 8. Treatment of compounds of formula 8 with 2,4-dichloro-5-methoxyaniline in the presence of pyridine hydrochloride provides the key 7-fluro intermediates 9.

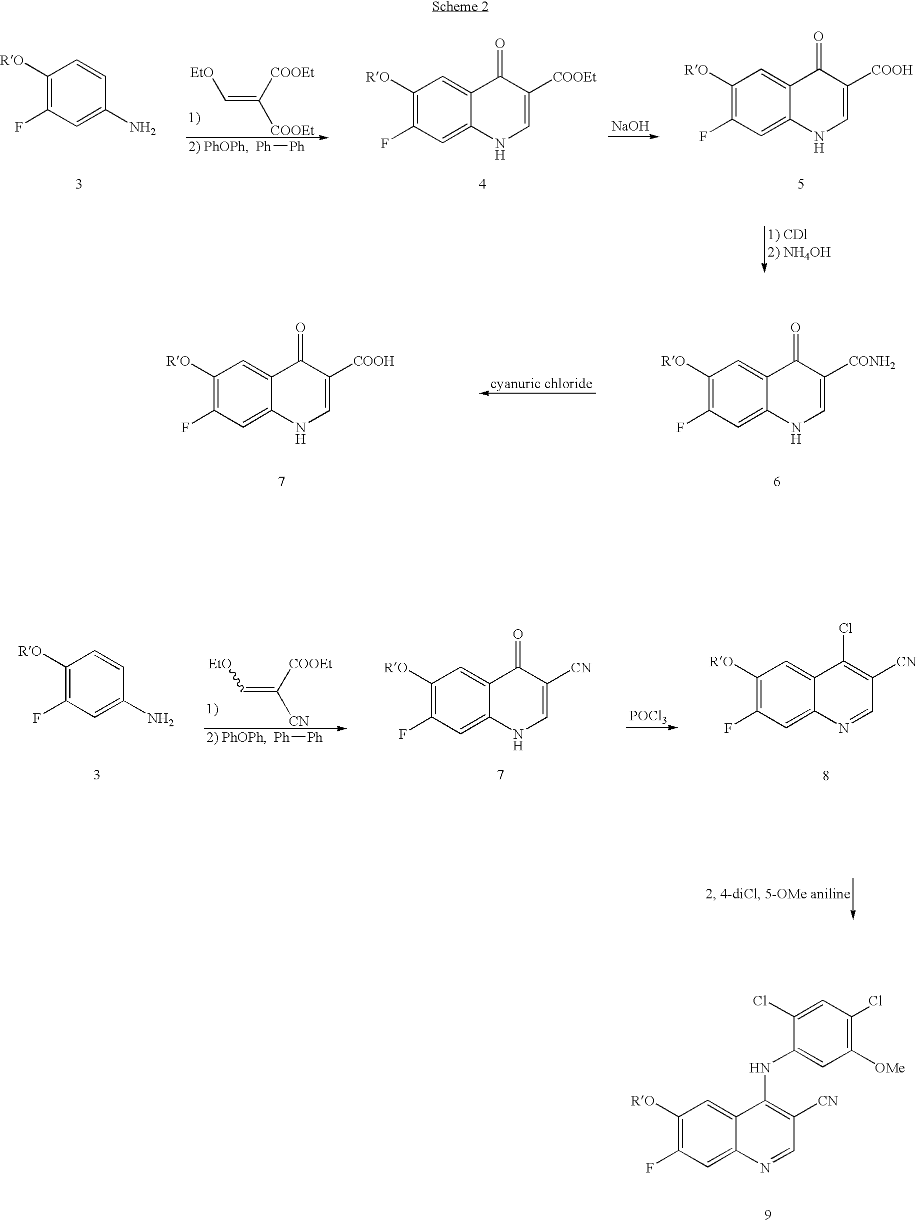

An alternate route to the compound of formula 8 where R′ is Et is shown in Scheme 3. Using the conditions of Scheme 2, 4-benzyloxy-3-fluoroaniline is converted into the compound of formula 10. Removal of the benzyl group with thioanisole and trifluoroacetic acid provides the 6-hydroxy derivative of formula 11. Treatment of 11 with triphenyl phosphine, diethylazodicarboxylate and ethanol gives the compound of formula 8 wherein R′ is ethyl.

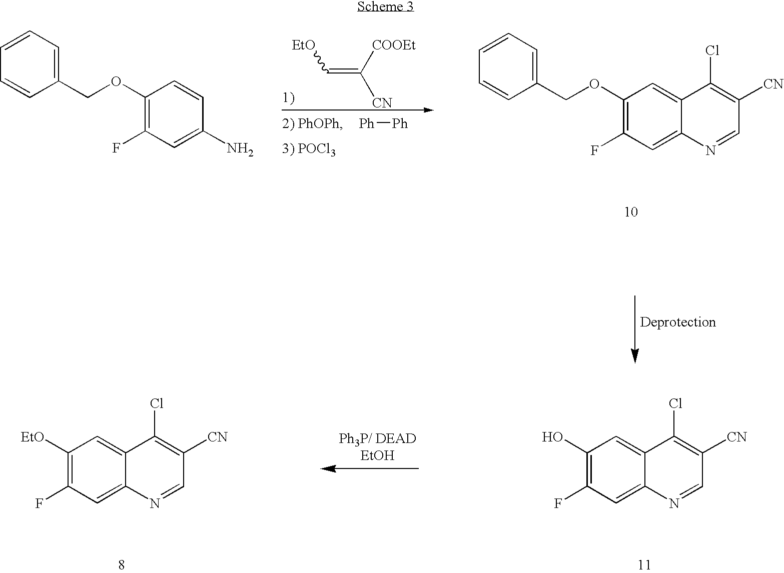

As shown in Scheme 4 reaction of compounds of formula 9 with an alcohol of formula 12 in the presence of a base such as sodium or sodium hydride provides the compounds of the invention of Formula I. This reaction can be run in the presence of a cosolvent such as dimethylformamide or dimethyl sulfoxide at optimal temperatures of 120° C. to 140° C.

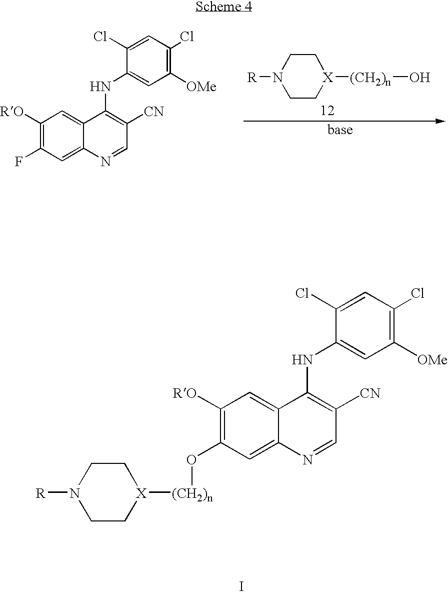

Compounds of the present invention were evaluated in several standard pharmacological tests that showed that compounds of the present invention inhibit Src kinase and are useful for the prevention of vascular permeability.

Src Kinase Assay

Inhibitors of Src (partially purified enzyme preparation purchased from Upstate Biotechnologies, Lake Placid, N.Y.) tyrosine kinase activity are analyzed in an ELISA format. The Boehringer Mannheim Tyrosine Kinase Assay Kit (Roche Diagnostics, Basel, Switzerland) with a cdc2 substrate peptide containing Tyr15 is used for the assay. Horseradish Peroxidase (HRP)-conjugated anti-phosphotyrosine is used to detect phosphorylated peptide via a color reaction.

Reaction conditions: Five microliter aliquots of each compound prepared fresh at the time of the assay are added as a solution in 10 mM HEPES pH 7.5, 10% DMSO to the reaction well. Thirty-five microliters of reaction mix containing Src, buffer and peptide/bovine serum albumin mix are added to the compound wells and incubated at 30° C. for 10 minutes (reaction buffer: 50 mM TrisHCl pH 7.5, 10 mM MgCl₂, 0.1 mM EGTA, 0.5 mM Na₃VO₄). The reaction is started by addition of 10 microliters of ATP (500 μM), incubated at 30° C. for 1 hour, and stopped by addition of 20 microliters of 0.5M EDTA. The reaction mixture with the phosphorylated peptide is then transferred to a streptavidin-coated microtiter plate and allowed to bind for 20 minutes. Unbound peptide and reaction mixture is decanted and the plate is washed with PBS six times. HRP-conjugated phosphotyrosine antibody supplied in the kit is incubated with the plate for one hour, then decanted. The plate is again washed with PBS six times. Substrate is added and absorbance at 405 nm is measured.

Alternatively, the assay performed essentially as described except a Delfia format (Perkin-Elmer) is used and Europium-conjugated phosphotyrosine antibody was used instead of HRP-conjugated phosphotyrosine antibody, Pierce Superblock was used in place of bovine serum albumin and 6 washes were employed after the kinase reaction and antibody binding. Europium fluorescence was used to monitor the extent of reaction.

Activity is determined as % inhibition as calculated by the formula: (1−Abs/Abs(max))×100=% inhibition. Where multiple concentrations of the test agent are used, an IC₅₀ (concentration which gives 50% inhibition) can be determined. As shown in Table 1, compounds of the invention inhibit src kinase in vitro.

Anchorage Independent Src-transformed Fibroblast Proliferation Assay

Rat2 fibroblasts stably transformed with a plasmid containing a CMV promotor controlled v-Src/Hu c-Src fusion gene in which the catalytic domain of human c-Src was inserted in place of the v-Src catalytic domain in the v-Src gene as follows: Cloning and plasmid constructions. The Prague C v-Src gene from pSrcHis (Wendler and Boschelli, Oncogene 4: 231-236; 1989) was excised with NcoI and BamHI, treated with T4 DNA polymerase, and cloned into the RI site of pTRE (Clontech) that had been rendered flush by treatment with T4 DNA polymerase. The PrC v-Src::hu c-Src fusion was created by replacing the BgI2-XbaI fragment encoding the carboxyl terminal ˜250 amino acids of v-Src with the BgI2-XbaI fragment containing the v-Src::huc-Src fusion fragment (below). A partial clone of human c-Src was amplified from a breast cDNA library (InVitrogen) using the oligonucleotide pair 5′-CGCCTGGCCAACGTCTGCCCCACGTCCAAGCCGCAGACTCAGGGCCTG-3′(SEQ ID NO: 1) and 5′-CCAACACACAAGCAGGGAGCAGCTGGGCCTGCAGGTACTCGAAGGTGGGC-3′(SEQ ID NO: 2) and cloned into pCRScript (Stratagene). The catalytic domain of human c-Src in this clone was amplified with these oligonucleotides (fuses v-src nucleotide 734 to human c-Src nucleotide 742 and human c-Src nucleotide 1551 to v-src nucleotide 1543 in the v-Src and human c-Src ORFs). Two v-Src sequences were amplified by PCR (198 base pair v-src 5′ fragment: 5′-GTGCCTATTGCCTCTCCGTTTCTGAC-3′(SEQ ID NO: 3) (primer 1) to 5′-ACGTGGGGCAGACGTTGGCCAGGCG-3′) (SEQ ID NO: 4) (252 base pair 3′ v-src fragment, 5′-CAGCTGCTCCCTGCTTGTGTGTTGG-3′ (SEQ ID NO: 5) (residues 1543-1567 in v-src ORF) to 5′-ATGAATTCTCTAGAGGAAGACGCCATCATATTCCAAGCAG-3′(SEQ ID NO: 6) residues 1769-1794 from v-src ATG with XbaI and EcoRI restriction sites added (primer 4)). Primers 1 and 4 were used to generate a three-fragment PCR amplification and fusion of the v-Src::human c-Src fusion fragment and the 5′ and 3′ fragments amplified from the Prague C v-Src gene and 3′ untranslated region from Rous sarcoma virus. This reaction creates an in-frame v-Src::human c-Src gene fusion (amino acid residue V244 of v-Src to C248 of human c-Src on the amino terminal side and A517 of human c-Src to Q515 of v-Src). This gene fusion fragment encodes the carboxyl terminal one-third of the v-Src SH2 domain and SH2-catalytic domain linker fused to the human c-Src catalytic domain flanked by the v-Src carboxyl-terminal tail. A naturally occurring BgI2 site near the 5′ end of the fusion fragment and the engineered XbaI site at the 3′ end of the fragment were used to excise fragment for creation of the full-length v-Src::human c-Src fusion gene as described above. The integrity of the constructs was confirmed by DNA sequencing. Similar methods were used to clone this gene into other expression plasmids such as pIRES (Clontech) for use in these studies.

These transformed Rat2 fibroblasts are used for the measurement of src dependent suspension growth.

Ultra-low cluster plates (Corning Costar, Acton, Mass.) are seeded with 10,000 cells per well on Day 1. Alternatively, Ultra-low cluster plates (Costar 3474) treated with Sigmacote (Sigma, St. Louis, Mo.), rinsed with 70% ethanol, after drying in the hood, are seeded with 5000 cells. Compound is added in serial two-fold dilutions from 10 micromolar to 0.009 micromolar on Day 2 and MTS reagent (Promega, Madison, Wis.) is added on Day 5 (100 microliters of MTS/medium mix+100 microliters of medium already on the cells and the absorbance is measured at 490 nm. The results are analyzed as follows to yield an IC₅₀ for proliferation (micromolar units) as follows: % inhibition=(Abs490 nm sample−blank)/(Abs490 nm no cmpd control−blank)×100%. As shown in Table 1, compounds of the present invention inhibit src dependent cell proliferation.

TABLE 1
Inhibition of Src enzymatic and cellular activity

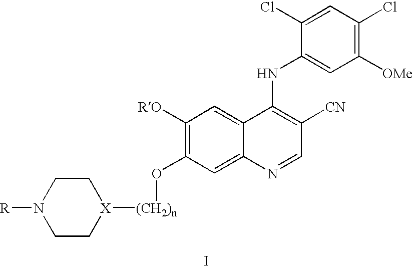

| | | | | | Src | Src |
|---|---|---|---|---|---|---|
| | | | | | enzyme | cells |
| Example | X | R | n | R′ | IC₅₀ nM | IC₅₀ nM |
| 1 | N | Me | 3 | Me | 1.2 | 100 |
| 2 | N | Et | 3 | Me | 0.77 | 130 |
| 3 | N | Me | 2 | Me | 4.0 | 380 |
| 4 | N | Et | 2 | Me | 3.6 | 600 |
| 5 | CH | Me | 1 | Me | 2.0 | 320 |
| 6 | CH | Me | 2 | Me | 1.9 | 210 |
| 7 | CH | Me | 3 | Me | 1.4 | 100 |
| 8 | CH | Et | 1 | Me | 2.1 | 170 |
| 9 | N | Me | 3 | Et | NT | 86 |
| 10 | CH | Me | 1 | Et | 2.1 | 176 |
| 11 | N | Et | 3 | Et | 0.85 | 160 |
| 12 | CH | Me | 3 | Et | 1.4 | 96 |
| 13 | N | Me | 2 | Et | 1.5 | 146 |
| 14 | CH | Me | 2 | Et | 1.9 | 267 |
| 15 | N | n-Pr | 3 | Me | 1.1 | 160 |

IP Administration of Example 1 Provides Neuroprotection in Transient Model of Focal Ischemia

Example 1 was tested in a rat model of transient focal ischemia. Wistar rats were subjected to a 90 min occlusion of the middle cerebral artery (MCA) using an intraluminal suture approach as described by Longa et al., Stroke 1989, 20:84 followed by reperfusion for 48 hours. Eighty-five minutes after the initial onset of ischemia, animals received compound of Example 1(1.5, 5, 15, or 45 mg/kg ip). Following reperfusion, the animals were evaluated over a 48 hour period for neurological function deficit and weight loss/gain. Infarct size was measured following sacrifice at 48 hours post MCA occlusion. Example 1 at doses of 5 and 45 mg/kg significantly improved recovery from stroke-induced neurological deficits. Reductions in the volume of infarcted brain tissue were observed at most doses of Example 1 but statistical significance was achieved only at the 45 mg/kg ip dose. Improvement in body weight recovery was observed in animals treated with Example 1.

IV Administration of Example 1 Provides Neuroprotection in Transient Model of Focal Ischemia

Wistar rats were subjected to a 90 min occlusion of the middle cerebral artery (MCA) using an intraluminal suture approach as described by Longa et al., Stroke 1989, 20:84 followed by reperfusion for 48 hours. Thirty minutes after MCA occlusion, an intravenous formulation of Example 1 in 20 mM citrate/0.85% saline, pH 3 was administered at doses of 3, 10 and 30 mg/kg (iv). Following reperfusion, the animals were evaluated over a 48 hour period for neurological function deficit and weight loss/gain. Brain tissue infarction volume by were reduced by 22%, 53% and 42%, respectively. Post-stroke weight loss was also significantly reduced. In addition, as shown in Table 2, stroke-induced neurological deficits were significantly reduced at all three doses. Thus, compounds of the present invention provide neuroprotection following focal ischemia.

TABLE 2

| | Mean Motor | P Value | Mean Motor | P Value |
|---|---|---|---|---|
| | Deficit Score at | (from | Deficit Score at | (from |
| Treatment | 24 hrs | control) | 48 hrs | control) |
| Vehicle- | 4.55 ± 0.16 | N/A | 4.27 ± 0.14 | N/A |
| control | | | | |
| 3 mg/kg | 3.83 ± 0.3* | p = 0.007 | 3.25 ± 0.37* | P = 0.0001 |
| 10 mg/kg | 4.08 ± 0.08 | p = 0.09 | 3.67 ± 0.22* | P = 0.016 |
| 30 mg/kg | 4.08 ± 0.23 | p = 0.09 | 3.67 ± 0.28* | P = 0.016 |

Therapeutic Window

In this model of transient focal ischemia three studies were conducted to examine therapeutic window. Wistar rats were subjected to 90 minute occlusion of the MCA followed by reperfusion as described above. A single bolus of 10 mg/kg Example 1 was administered at 30 minutes, 90 minutes, 3 hours, 4 hours, 5 hours and 6 hours post stroke. Volume of infarcted tissue was measured by histological staining. Brain tissue infarction was statistically reduced (as a % of vehicle treated) with a single 10 mg/kg dose of Example 1 administered between 30 min and 4 hours after the ischemic injury. Statistically significant protection from neurological deficits (as a percent of vehicle treated) was achieved with a single 10 mg/kg dose of Example 1 administered up to 5 hours post-stroke, and statistically significant protection from ischemia-induced weight loss (as a % of vehicle treated) was achieved with a single 10 mg/kg dose of Example 1 up to 5 hours post stroke. Thus, compounds of the present invention exhibit a superior therapeutic window compared to presently available treatments.

Post Ischemic Vascular Permeability

Wistar rats were subjected to a 90 min occlusion of the middle cerebral artery (MCA) using an intraluminal suture approach as described by Longa et al., Stroke 1989, 20:84 followed by reperfusion for 24 hours. Compound of Example 1 was administered as a single IV bolus at 30 minutes after onset of ischemia at 3, 10 and 30 mg/kg (iv). Two hours before sacrifice animals received an IV injection of 2% Evans Blue in saline. Brains were perfused with saline and the striatum dissected. Evans Blue was extracted and quantified by spectrofluorometer based on external standards. Vascular permeability in the ischemic striatum was reduced as evidenced by a 60% decrease of Evans Blue extravasation. Thus, compounds of the present invention reduce vascular permeability associated with ischemic injury.

Permanent Focal Ischemia

Example 1 was also evaluated in two rat models of permanent focal ischemia. In a model of extreme severity (intraluminal suture occlusion of internal carotid artery) and a relatively short outcome (28 hours) little or no effect was shown.

In a model producing extensive infarction to sensorimotor cortex with quantitative assessment of neurological deficits for 21 days post-stroke, compound of Example 1 provided significant improvement in the neurological outcome after stroke. Wistar rats (n=5 per group) were subjected to focal ischemic stroke model that results in a extensive ischemia to the sensorimotor cortex substantially as described by Chen et al., (Stroke 17:738, 1986). Example 1 or vehicle was administered as an IV bolus at 10 mg/kg at 90 minutes post-induction of stroke, 4 hours later, and at 24 and 28 hours later (total dose 40 mg/kg). Animals were evaluated for sensorimotor deficits (postural reflex, visual and tactile forelimb placement and hindlimb placement tests) on days 1, 2, 4, 7, 9,11, 14, 16, 18 and 21 after induction of ischemia. Results were evaluated by Generalized Regression Model to determine statistical significance of the differences between slopes and to compare final neurological outcome after 21 days. By Day 21, there was statistically significant improvement in Behavioral Score for subjects treated with Example 1 as compared to a control group. Thus, compounds of the present invention provide long-term improvement of neurological deficits.

Vascular permeability due to disease, injury, or other trauma, may occur in a variety of tissues and organs including organs of the central nervous system, cardiopulmonary system, gastrointestinal system and renal system. Compounds of the present invention are useful for inhibiting vascular permeability caused by disease, injury, or other trauma. In particular, vascular permeability may be inhibited in cerebral and spinal tissue following cerebrovascular events. Vascular permeability is a major cause of vascular leakage and/or edema following a cerebrovascular event and often leads to neurological disorders and disabilities. Cerebrovascular events including, but not limited to transient and acute ischemic events, may be treated in accordance with the present invention. Acute events include, but are not limited to, stroke, head trauma, spinal trauma, general anoxia, hypoxia including fetal hypoxia, hypoglycemia, hypotension as well as similar injuries seen during procedures from embole, hyperfusion and hypoxia. Stroke includes, but is not limited to focal and global ischemia, transient cerebral ischemic attacks, and other cerebral vascular problems accompanied by cerebral ischemia. The instant invention would also be useful in a range of cerebrovascular events including cerebral hemmorhage, infarction due to embolism or thrombosis of the intra- or extra cranial arteries, perinatal asphyxia, in cardiac arrest and status epilepticus, especially where blood flow to the brain in halted for a period of time. Cerebrovascular events associated with vascular leakage also include infections, including, but not limited to encephalitis and meningitis associated with neuroinflammation, which, through vascular leakage propagate injury to surrounding tissues. Systemic disease such as diabetes, multiple sclerosis, kidney disease and atherosclerosis may also result in increased vascular permeability. Compounds of the present invention are also useful for inhibiting vascular permeability triggered by any local tissue/organ ischemic (hypoxic) event outside of the central nervous system, including, but not limited to myocardial ischemia and ischemic bowel disease.

Compounds of the present invention provide neuroprotection in a patient. Neuroprotection, as used herein, refers to the protection of neural cells against cell death or apoptosis. One measure of the extent of cell death or apoptosis is infarct volume; the volume of necrotic or dead brain tissue. Imaging techniques and the patient's clinical status can be used to assess infarct volume following an ischemic event. Compounds of the present invention reduce infarct volume of a patient as compared to typical infarct volume experienced in similar ischemic events in the absence of agents of the present invention.

Compounds of the present invention prevent, reduce or inhibit neurodegeneration and/or neurotoxicity associated with vascular permeability that result in symptoms including, but not limited to, visual impairment, speech impairment, memory impairment, cognitive impairment or dysfunction, and motor impairment including, but not limited to, paralysis. Neurological deficits resulting from injury or disease described above may be inhibited or prevented in accordance with the present invention. Thus, the present invention provides methods of treating, preventing, inhibiting or alleviating conditions associated with vascular leakage or permeability listed above in a mammal, preferably in a human, the methods comprising providing a pharmaceutically effective amount, and in particular a vascular permeability inhibiting amount, of a compound of this invention to the mammal, and in particular a human patient, in need thereof.

Also encompassed by the present invention are pharmaceutical compositions for treating or modulating vascular permeability comprising at least one compound of Formula I, mixtures thereof, and or pharmaceutical salts thereof, and a pharmaceutically acceptable carrier therefore. Such compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

Liquid carriers may be used in preparing solutions, suspensions, emulsions, syrups and elixirs including intravenous solutions. The active ingredient of this invention can be dissolved or suspended in a pharmaceutically acceptable liquid carrier such as water, organic solvent, or a mixture of both. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, osmo-regulators, antioxidants and antifoaming agents.

Suitable examples of liquid carriers for oral, intravenous and parenteral administration include water (particularly containing additives as above e.g. cellulose derivatives, preferably sodium carboxymethyl cellulose solution), saline, dextrose solutions, dextrose-saline and dextrose-water solutions, alcohols (including monohydric alcohols and polyhydric alcohols e.g. glycols) and their derivatives. Liquid carriers are used in sterile form for parenteral and intravenous administration. PH of liquid formulations may be adjusted in some cases by the addition of HCl, sodium hydroxide, and phosphoric acid. Preferably compositions of the present invention are liquid pharmaceutical compositions which are sterile solutions or suspensions in an iso-osmotic, physiologically compatible buffered system.

Liquid pharmaceutical compositions of the present invention can be administered by, for example, intramuscular, intraperitoneal, intravenous, or subcutaneous injection. Pharmaceutical compositions of the present invention are preferably administered to a patient by intraperitoneal or intravenous injection. Most preferably, the composition is administered intravenously such as by intravenous bolus injection, intravenous i.v. drip, repeated slow bolus administration or infusion.

Oral administration may be either liquid or solid composition form. The compounds of this invention may also be administered orally or parentally, neat or in combination with conventional pharmaceutical carriers. Applicable solid carriers can include one or more substances which may also act as flavoring agents, lubricants, solubilizers, suspending agents, fillers, glidants, compression aids, binders or tablet-disintegrating agents or an encapsulating material. In powders, the carrier is a finely divided solid, which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Preferably the pharmaceutical composition is in unit dosage form, e.g. as tablets, capsules, powders, solutions, suspensions, emulsions, granules, suppositories, ampule, or bolus. In such form, the composition is sub-divided in unit dose containing appropriate quantities of the active ingredient; the unit dosage forms can be packaged compositions, for example packeted powders, lyophilyzed powder or cake in ampoules or vials, or vials, ampoules, prefilled syringes or sachets containing liquids. The unit dosage form can be, for example, capsule or tablet itself, or it can be the appropriate number of any such compositions in package form.

The dose provided to a patient will vary depending upon what is being administered, the purpose of the administration, such as prophylaxis or therapy, and the state of the patient, the manner of administration, and the like. A “therapeutically effective amount” is an amount sufficient to cure or ameliorate symptoms of a disease or injury. Generally, a single dose (or dosage form) will contain from about 1 mg/kg to about 30 mg/kg, and more preferably from about 1 mg/kg to about 10 mg/kg of compound of the present invention. It is expected that some patients will receive multiple doses. The dosage to be used in the treatment of a specific case must be subjectively determined by the attending physician. The variables involved include the specific condition and the size, age and response pattern of the patient.

The present invention provides advantages over previously known treatments for stroke and other conditions associated with vascular permeability. In particular, while it is preferable to treat patients as soon as possible after an ischemic injury, compounds of the present invention may be effective in preventing neurodegeneration and development of neurological deficits in some patients when administered even up to about 18-24 hours after ischemic injury. Furthermore, treatment may continue and improvement in a patient's prognosis may result from continuous or repeated administration of compound of the present invention for up to about 72 hours or longer following ischemic injury.

Provide as used herein means either directly administering a compound or composition of the present invention, or administering a prodrug, derivative or analog which will form an equivalent amount of the active compound or substance within the body.

The present invention includes prodrugs of compounds of Formula I. “Prodrug”, as used herein means a compound which is convertible in vivo by metabolic means (e.g. by hydrolysis) to a compound of Formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991), Bundgaard, et al., Journal of Drug Deliver Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975).

This invention will be more fully described in conjunction with the following specific examples which are not to be construed as limiting the scope of this invention.

### REFERENCE EXAMPLE 1

### Ethyl 7-fluoro-6-methoxy-4-oxo-1,4-dihydro-3-guinolinecarboxylate

A mixture of 3-fluoro-4-methoxyaniline (3.00 g, 21.26 mmol) and diethyl ethoxymethylene malonate (4.59 g, 21.26 mmol) was heated at 110° C. for 1 hour then cooled to room temperature. Hexane was added and the solids were collected by filtration. This material was suspended in 45 mL of a 3:1 mixture of diphenyl ether:biphenyl and the mixture was heated at reflux for 2 hours to provide a brown solution. The reaction mixture was cooled to room temperature and hexane was added. The resultant solid was collected by filtration washing with hexane to provide 2.62 g of ethyl 7-fluoro-6-methoxy-4-oxo-1,4-dihydro-3-quinolinecarboxylate as a white solid,

mp >300° C.

MS 265.9 (M+H)+.

Analysis for C₁₃H₁₂FNO₄.

Calcd: C, 58.87; H, 4.56; N, 5.28. Found: C, 58.66; H, 4.16; N, 5.14.

### REFERENCE EXAMPLE 2

### 7-Fluoro-6-methoxy-4-oxo-1,4-dihydro-3-guinolinecarboxylic acid

A mixture of ethyl 7-fluoro-6-methoxy-4-oxo-1,4-dihydro-3-quinolinecarboxylate (2.2 g, 8.30 mmol) and 13.2 mL of 1 N sodium hydroxide and 40 mL of ethanol was heated at reflux for 3 hours then cooled to room temperature. Water was added and the mixture was acidified with acetic acid. The resultant solid was collected by filtration washing with water to provide 1.90 g of 7-fluoro-6-methoxy-4-oxo-1,4, -dihydro-3-quinolinecarboxylic acid as a white solid, mp 265-267° C.

MS 238.1 (M+H)+.

Analysis for C₁₁H₈FNO₄-1.2 H₂O.

Calcd: C, 51.04; H, 4.03; N, 5.41. Found: C, 50.98; H, 3.95; N, 5.33.

### REFERENCE EXAMPLE 3

### 7-Fluoro-6-methoxy-4-oxo-1,4-dihydro-3-quinolinecarboxamide

A mixture of 7-fluoro-6-methoxy-4-oxo-1,4-dihydro-3-quinolinecarboxylic acid (1.0 g, 4.21 mmol) and 1,1′-carbonyldiimidazole (1.51 g, 9.28 mmol) in 14 mL of N,N-dimethylformamide was heated at 65° C. for 2 hours then cooled to room temperature and poured into 200 mL of aqueous ammonium hydroxide on an ice water bath. The solution was allowed to stir at room temperature overnight and then concentrated to a small volume. Ice cold water was added followed by acidification with acetic acid. The resultant solid was collected by filtration washing with water to provide 821 mg of 7-fluoro-6-methoxy-4-oxo-1,4-dihydro-3-quinolinecarboxamide as a white solid, mp >300° C.

MS 236.8 (M+H)+.

Analysis for C₁₁H₈FN₂O₃-0.2 H₂O.

Calcd: C, 55.09; H, 3.94; N, 11.68. Found: C, 55.00; H, 3.63; N, 11.49.

### REFERENCE EXAMPLE 4

### 7-Fluoro-6-methoxy-4-oxo-1,4-dihydro-3-quinolinecarbonitrile

A mixture of 7-fluoro-6-methoxy-4-oxo-1,4,-dihydro-3-quinolinecarboxamide (700 mg, 3.0 mmol) and cyanuric chloride (341 mg, 1.65 mmol) in 15 mL of N,N-dimethylformamide was heated at 65° C. for 6 hours then cooled to room temperature and an additional 206 mg of cyanuric chloride was added. The mixture was heated at 65° C. for 4 hours then stirred overnight at room temperature. The reaction mixture was poured into ice water and neutralized with saturated sodium bicarbonate. The solids were collected by filtration washing with water and hexane to provide 610 mg of crude product. Purification by flash column chromatography eluting with a gradient of 3% methanol in dichloromethane to 10% methanol in dichloromethane, provided 272 mg of 7-fluoro-6-methoxy-4-oxo-1,4-dihydro-3-quinolinecarbonitrile, mp 147-149° C.

MS 216.8 (M−H)−.

Analysis for C₁₁H₇FN₂O₂-0.1 dichloromethane.

Calcd: C, 58.80; H, 3.19; N, 12.36. Found: C, 59.06; H, 2.96; N, 11.97.

### ALTERNATIVE ROUTE TO REFERENCE EXAMPLE 4

### 7-Fluoro-6-methoxy-4-oxo-1,4-dihydro-3-quinolinecarbonitrile

A mixture of 3-fluoro-4-methoxyaniline (15.31 g, 108 mmol) and ethyl (ethoxymethylene)cyanoacetate (18.36 g, 108 mmol) in toluene was heated at 100-110° C. for 4.5 hours then cooled to room temperature. A 1:1 mixture of hexane and ethyl acetate was added and the mixture was cooled on an ice bath. The solids were collected washing with hexane to provide a first crop of 26.10 g and a second crop of 1.24 g. A 2.0 g portion of this material was added to 18 mL of a 3:1 mixture of diphenyl ether:biphenyl that was heated to reflux. This mixture was heated at reflux for 4 hours then cooled and poured into hexane. The solids were collected by filtration and washed with ethyl acetate and hexane to provide 624 mg of 7-fluoro-6-methoxy-4-oxo-1,4,-dihydro-3-quinolinecarbonitrile as a brown solid. The filtrate was concentrated, the residue was dissolved in ethyl acetate and hexane was added. The resultant solid was collected by filtration to give 1.07 g of 7-fluoro-6-methoxy-4-oxo-1,4-dihydro-3-quinolinecarbonitrile as a yellow solid.

### REFERENCE EXAMPLE 5

### 4-Chloro-7-fluoro-6-methoxy-3-quinolinecarbonitrile

A mixture of 7-fluoro-6-methoxy-4-oxo-1,4-dihydro-3-quinolinecarbonitrile (1.0 g, 4.59 mmol) and 14 g of phosphorous oxychloride was heated at reflux for 30 minutes then concentrated in vacuo. The residue was partitioned between aqueous sodium bicarbonate and ethyl acetate. The organic layer was dried over magnesium sulfate, filtered and concentrated on to silica gel. Purification by flash column chromatography eluting with a gradient of 1:5 ethyl acetate:hexane to 1:1 ethyl acetate:hexane, provided 631 mg of 4-chloro-7-fluoro-6-methoxy-3-quinolinecarbonitrile, mp 160-162° C.

MS 236.9 (M+H)+.

Analysis for C₁₁H₆ClFN₂O.

Calcd: C, 55.83; H, 2.56; N, 11.84. Found: C, 55.66; H, 2.84; N, 11.91.

### REFERENCE EXAMPLE 6

### 4-[(2,4-Dichloro-5-methoxyphenyl)amino]-7-fluoro-6-methoxy-3-quinolinecarbonitrile

A mixture of 4-chloro-7-fluoro-6-methoxy-3-quinolinecarbonitrile (4.12 g , 18 mmol) 2,4-dichloro-5-methoxyaniline (4.56 g, 24 mmol) (Theodoridis, G.; Pestic. Sci. 1990, 30, 259) and pyridine hydrochloride (2.31 g, 19.9 mmol) in 45 mL of 2-ethoxyethanol was heated at 120° C. for 3 hours then cooled to room temperature. The reaction mixture was added to aqueous sodium bicarbonate and stirred for 20 minutes. The solids were collected by filtration to provide 4.89 g of 4-[(2,4-dichloro-5-methoxyphenyl)amino]-7-fluoro-6-methoxy-3-quinolinecarbonitrile, mp >260° C.

HRMS theory 392.03634; found 392.03556 (M+H)+.

Analysis for C₁₈H₁₂Cl₂FN₃O₂-2.0 H₂O.

Calcd: C, 50.48; H, 3.77; N, 9.81. Found: C, 50.41; H, 2.82; N, 9.78.

### REFERENCE EXAMPLE 7

### 6-Benzyloxy-7-fluoro-4-oxo-1,4-dihydro-3-quinolinecarbonitrile

A mixture of 4-benzyloxy-3-fluoroaniline (6.06 g, 27.9 mmol) (U.S. Pat. No. 5,622,967) and ethyl (ethoxymethylene)cyanoacetate (5.08 g, 30.0 mmol) was heated at 120° C. for 45 minutes then cooled to room temperature. This solid was added in portions to a 3:1 mixture of diphenyl ether:biphenyl at 245° C. This mixture was heated at 245° C. for 3 hours then cooled and the solids were collected by filtration, washing with hexane and diethyl ether to provide 2.60 g of 6-benzyloxy-7-fluoro-4-oxo-1,4-dihydro-3-quinolinecarbonitrile, mp >250° C.

MS 293.1 (M−H)−.

### REFERENCE EXAMPLE 8

### 6-Benzyloxy-4-chloro-7-fluoro-3-quinolinecarbonitrile

A mixture of 6-benzyloxy-7-fluoro-4-oxo-1,4-dihydro-3-quinolinecarbonitrile (645 mg, 2.19 mmol) and 10 mL of phosphorous oxychloride was heated at 115° C. for 1.5 hours then concentrated in vacuo. The residue was treated with ice cold aqueous ammonium hydroxide and the resultant solid was collected by filtration. Purification by flash column chromatography eluting with a gradient of 1% ethyl acetate in hexane to 6% ethyl acetate in hexane, provided 284 mg of 6-benzyloxy-4-chloro-7-fluoro-3-quinolinecarbonitrile, mp 159-160° C.

MS 313.13 (M+H)+.

Analysis for C₁₇H₁₀ClFN₂O.

Calcd: C, 65.15; H, 3.06; N, 8.82. Found: C, 65.29; H, 3.22; N, 8.96.

### REFERENCE EXAMPLE 9

### 4-Chloro-7-fluoro-6-hydroxy-3-quinolinecarbonitrile

A mixture of 6-benzyloxy-4-chloro-7-fluoro-3-quinolinecarbonitrile (733 mg, 2.34 mmol) and 1 mL of thioanisole in 12 mL of trifluoroacetic acid was heated at reflux for 9 hours then concentrated in vacuo. The residue was treated with ice water and then basified to pH 9-10 by the addition of aqueous ammonium hydroxide. The resultant solid was collected by filtration and washed with diethyl ether. The filtrate was extracted with 10% methanol in ethyl acetate. The organic layer was dried over sodium sulfate, filtered and concentrated in vacuo. The residue was combined with the solid obtained initially, and this material was dissolved in 5% methanol in ethyl acetate and absorbed onto silica gel. Purification by flash column chromatography eluting with a gradient of hexane to increasing amounts of ethyl acetate in hexane to 5% methanol in ethyl acetate provided 260 mg of 4-chloro-7-fluoro-6-hydroxy-3-quinolinecarbonitrile, mp >250° C.

MS 220.9 (M−H)−.

Analysis for C₁₀H₄ClFN₂O.

Calcd: C, 53.96; H, 1.81; N, 12.58. Found: C, 54.23; H, 2.02; N, 12.06.

### REFERENCE EXAMPLE 10

### 4-Chloro-6-ethoxy-7-fluoro-3-quinolinecarbonitrile

To a 0° C. mixture of 4-chloro-7-fluoro-6-hydroxy-3-quinolinecarbonitrile (185 mg, 0.83 mmol), triphenylphosphine (392 mg, 1.49 mmol) and ethanol (153 mg, 3.32 mmol) in 15 mL of tetrahydrofuran was added diethylazodicarboxylate (260 mg, 1.80 mmol). The reaction mixture was kept at 0° C. for 45 minutes then stirred at room temperature overnight. The reaction mixture was concentrated in vacuo and purified by flash column chromatography eluting with a gradient of 1% ethyl acetate in hexane to 5% ethyl acetate in hexane to provide 4-chloro-6-ethoxy-7-fluoro-3-quinolinecarbonitrile, mp 165-166° C.

MS 251.0 (M+H)+.

Analysis for C₁₂H₈ClFN₂O.

Calcd: C, 57.50; H, 3.22; N, 11.18. Found: C, 57.24; H, 3.41; N, 11.09.

### REFERENCE EXAMPLE 11

### 4-[(2,4-Dichloro-5-methoxyphenyl)amino]-6-ethoxy-7-fluoro-3-quinolinecarbonitrile

Following the procedure of Reference Example 6, a mixture of 4-chloro-6-ethoxy-7-fluoro-3-quinolinecarbonitrile (197 mg, 0.78 mmol), 2,4-dichloro-5-methoxyaniline (220 mg, 1.14 mmol) and pyridine hydrochloride (120 mg, 1.04 mmol) provided, after flash column chromatography eluting with a gradient of dichloromethane to 1% methanol in dichloromethane, 183 mg of 4-[(2,4-dichloro-5-methoxyphenyl)amino]-6-ethoxy-7-fluoro-3-quinolinecarbonitrile, mp 184-186° C.

MS 406.0 (M+H).

Analysis for C₁₉H₁₄Cl₂FN₃O₂-0.5 H₂O.

Calcd: C, 54.96; H, 3.64; N, 10.12. Found: C, 54.99; H, 3.59; N, 10.05.

### EXAMPLE 1

### 4-[(2,4-Dichloro-5-methoxyphenyl)amino]-6-methoxy-7-[3-(4-methyl-1-piperazinyl)propoxy]-3-quinolinecarbonitrile

A mixture of 7-[3-chloropropoxy]-4-[(2,4-dichloro-5-methoxyphenyl)amino]-6-methoxy-3-quinolinecarbonitrile (656 mg, 1.40-mmol) and sodium iodide (210 mg, 1.40 mmol) in 4 mL of N-methylpiperazine was heated at 80° C. for 20 h. The reaction mixture was concentrated in vacuo and partitioned between ethyl acetate and saturated aqueous sodium bicarbonate. The organic layer was washed with brine, dried over sodium sulfate, filtered and concentrated in vacuo. The residue was purified by column chromatography eluting with 30% methanol in dichloromethane. The fractions containing product were collected and concentrated in vacuo. Diethyl ether was added to the residue and the light pink solid was collected by filtration to provide 560 mg (75%) of 4-[(2,4-dichloro-5-methoxyphenyl)amino]-6-methoxy-7-[3-(4-methyl-1-piperazinyl)propoxy]-3-quinolinecarbonitrile: mp 116-120° C.; MS (ES) m/z 530.2, 532.2 (M+1).

### EXAMPLE 2

### 4-[(2,4-Dichloro-5-methoxyphenyl)amino]-7-[3-(4-ethyl-1-piperazinyl)propoxy]-6-methoxy-3-quinolinecarbonitrile

A mixture of 7-[3-chloropropoxy]-4-[(2,4-dichloro-5-methoxyphenyl)amino]-6-methoxy-3-quinolinecarbonitrile (3.50g, 7.50 mmol), sodium iodide (1.12 g, 7.50 mmol) and 4.8 mL of N-ethylpiperazine in 5 mL of ethylene glycol dimethyl ether was heated at 95° C. for 20 h. The reaction mixture was concentrated in vacuo and partitioned between ethyl acetate and saturated aqueous sodium bicarbonate. The organic layer was washed with saturated aqueous sodium bicarbonate, followed by brine, dried over sodium sulfate, filtered and concentrated in vacuo. Diethyl ether was added to the residue and the white solid was collected by filtration to provide 1.80 g (44%) of 4-[(2,4-dichloro-5-methoxyphenyl)amino]-7-[3-(4-ethyl-1-piperazinyl)propoxy]-6-methoxy-3-quinolinecarbonitrile: mp 102-104° C.; MS (ES) m/z 544.3, 546.4 (M+1).

### EXAMPLE 3

### 4-[(2,4-Dichloro-5-methoxyphenyl)amino]-6-methoxy-7-[2-(4-methyl-1-piperazinyl)ethoxy]-3-quinolinecarbonitrile

Prepared according to the method used for the preparation of Example 1 by the reaction of 7-[2-chloroethoxy]-4-[(2,4-dichloro-5-methoxyphenyl)amino]-6-methoxy-3-quinolinecarbonitrile and N-methylpiperazine: mp 165-167° C.; MS (ES) m/z 516.0, 518.2 (M+1).

### EXAMPLE 4

### 4-[(2,4-Dichloro-5-methoxyphenyl)amino]-7-[2-(4-ethyl-1-piperazinyl)ethoxy]-6-methoxy-3-quinolinecarbonitrile

Prepared according to the method used for the preparation of Example 1 by the reaction of 7-[2-chloroethoxy]-4-[(2,4-dichloro-5-methoxyphenyl)amino]-6-methoxy-3-quinolinecarbonitrile and N-ethylpiperazine: mp 101-105° C.; MS (ES) m/z 530.4, 532.4 (M+1).

### EXAMPLE 5

### 4-[(2,4-Dichloro-5-methoxyphenyl)amino]-6-methoxy-7-[(1-methylpiperidin-4-yl)methoxy]-3-quinolinecarbonitrile

To a solution of 4-[(2,4-dichloro-5-methoxyphenyl)amino]-7-fluoro-6-methoxy-3-quinolinecarbonitrile (600 mg, 1.53 mmol) and 1-methylpiperidine-4-methanol (395 mg, 3.06 mmol) in 10 mL of N,N-dimethylformamide at 135° C. was added sodium hydride (362 mg, 9.06 mmol) in portions. After 45 minutes the reaction mixture was poured into saturated sodium bicarbonate. After stirring for 15 minutes the solid was collected by filtration. The residue was purified by flash column chromatography, eluting with a gradient of 5% methanol in dichloromethane to 25% methanol in dichloromethane. Trituation with diethyl ether provided 396 mg of 4-[(2,4-dichloro-5-methoxyphenyl)amino]-6-methoxy-7-(1-methylpiperidin-4-yl)methoxy]-3-quinolinecarbonitrile, mp 200-202° C.

MS 501.3 (M+H)+.

Analysis for C₂₅H₂₆Cl₂N₄O₃-0.8H₂O.

Calcd: C, 58.21; H, 5.39; N, 10.86. Found: C, 58.19; H, 5.23; N, 10.67.

### EXAMPLE 6

### 4-[(2,4-Dichloro-5-methoxyphenyl)amino]-6-methoxy-7-[2-(1-methylpiperidin-4-yl)ethoxy]-3-quinolinecarbonitrile

A mixture of sodium hydride (128 mg, 3.2 mmol) and 1-methyl-4-piperidineethanol (180 mg, 1.25 mmol) [ EP 0581538] in 5 mL of N,N-dimethylformamide was heated at 110° C. for 1 hour. 4-[(2,4-Dichloro-5-methoxyphenyl)amino]-7-fluoro-6-methoxy-3-quinolinecarbonitrile (200 mg, 0.51 mmol) was added and the reaction mixture was heated at 135° C. for 5 hours. Over the next 4 hours an additional 128 mg of sodium hydride was added to the reaction mixture at 130° C. The reaction mixture was partitioned between ethyl acetate and water. The organic layer was dried over sodium sulfate, filtered and concentrated in vacuo. The residue was purified by preparative thin layer chromatography, eluting with 20% methanol in dichloromethane to provide 105 mg of 4-[(2,4-dichloro-5-methoxyphenyl)amino]-6-methoxy-7-[(2-(1-methylpiperidin-4-yl)ethoxy]-3-quinolinecarbonitrile, mp 190-191° C.

MS 515.19 (M+H)+.

Analysis for C₂₆H₂₈Cl₂N₄O₃-1.0 H₂O.

Calcd: C, 58.53; H, 5.67; N, 10.50. Found: C, 58.65; H, 5.57; N, 10.34.

Examples 7 and 8 are obtained analogously by the method of Example 5 and the corresponding alcohol.

### EXAMPLE 7

### 4-[(2,4-Dichloro-5-methoxyphenyl)amino]-6-methoxy-7-[3-(1-methylpiperidin-4-yl)propoxy]quinoline-3-carbonitrile

MP 144-145° C.; Mass spec. 529.2 (ES +).

### EXAMPLE 8

### 4-[(2,4-Dichloro-5-methoxyphenyl)amino]-7-[(1-ethylpiperidin-4-yl)methoxy]-6-methoxyquinoline-3-carbonitrile

MP 192-195° C.; Mass spec. 515.2 (ES +).

### EXAMPLE 9

### 4-[(2,4-Dichloro-5-methoxyphenyl)amino]-6-ethoxy-7-[3-(4-methylpiperazin-1-yl)propoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol), 3-(4-methyl-piperazin-1-yl)propanol (155 mg, 0.98 mmol) (WO 20047212) and sodium hydride (196 mg, 4.6 mmol) in 5 mL of N,N-dimethylformamide was heated at 125° C. for 3 hours. The reaction mixture was poured into saturated sodium bicarbonate and stirred for 1 hour. The aqueous solution was extracted with 10% methanol in dichloromethane. The organic layer was washed with brine, dried over magnesium sulfate and concentrated in vacuo. The residue was purified by preparative thin layer chromatography, eluting with 15% methanol in dichloromethane. Trituation with hexane provided 116 mg of 4-[(2,4-dichloro-5-methoxyphenyl)amino]-6-ethoxy-7-[3-(4-methylpiperazin-1-yl)propoxy]quinoline-3-carbonitrile as a light brown solid, mp 137-138° C.

MS 542.0 (M−H)−.

Analysis for C₂₇H₃₁Cl₂N₅O₃-0.6 H₂O.

Calcd: C, 58.40; H, 5.84; N, 12.61. Found: C, 58.31; H, 5.71; N, 12.43.

### EXAMPLE 10

### 4-[(2,4-Dichloro-5-methoxyphenyl)amino]-6-ethoxy-7-[(1-methylpiperidin-4-yl)methoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol), 1-methylpiperidine-4-methanol (188 mg, 0.98 mmol) (WO 20047212) and sodium hydride (196 mg, 4.6 mmol) in 5 mL of N,N-dimethylformamide was heated at 125° C. for 3 hours. The reaction mixture was poured into saturated sodium bicarbonate and stirred for 1 hour. The solid was collected by filtration, washed with water and dried in vacuo. The solid was purified by preparative thin layer chromatography, eluting with 15% methanol in dichloromethane. Trituation with diethyl ether provided 67 mg of 4-[(2,4-dichloro-5-methoxyphenyl)amino]-6-ethoxy-7-[(1-methylpiperidin-4-yl)methoxy]quinoline-3-carbonitrile as a light brown solid, mp 182-186° C.

MS 513.0 (M−H)−.

Analysis for C₂₆H₂₈Cl₂N₄O₃-1.4 H₂O.

Calcd: C, 57.76; H, 5.74; N, 10.36. Found: C, 57.65; H, 5.43; N, 10.15.

### EXAMPLE 11

### 4-[(2,4-Dichloro-5-methoxyphenyl)amino]-6-ethoxy-7-[3-(4-ethylpiperazin-1-yl)propoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol) and 3-(4-ethyl-piperazin-1-yl)propanol (241 mg, 0.98 mmol) ) in 5 mL of N,N-dimethylformamide was heated at 125° C. for 5 min. Sodium hydride (60%) (98 mg, 2.45 mmol) was added and the mixture was heated at 125° C. for 1 hour. Additional sodium hydride (98 mg, 2.45 mmol) was added and the mixture was heated at 125° C. for 2 hours. The reaction mixture was cooled to room temperature and poured into saturated sodium bicarbonate and stirred for 1 hour. The aqueous solution was extracted with 10% methanol in dichloromethane. The organic layer was dried over sodium sulfate and concentrated in vacuo. The residue was purified by preparative thin layer chromatography, developing with 12% methanol in dichloromethane. Trituation with diethyl ether and hexane provided 146 mg of 4-[(2,4-dichloro-5-methoxyphenyl)amino]-6-ethoxy-7-[3-(4-ethylpiperazin-1-yl)propoxy]quinoline-3-carbonitrile as a light brown solid, mp 127-130° C.

MS 558.3 (M+H)+.

Analysis for C₂₈H₃₃Cl₂N₅O₃-1.5 H₂O.

Calcd: C, 57.44; H, 6.20; N, 11.96. Found: C, 57.44; H, 6.24; N, 11.79.

### EXAMPLE 12

### 4-[(2,4-Dichloro-5-methoxyphenyl)amino]-6-ethoxy-7-[3-(1-methylpiperidin-4-yl)propoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol) and 3-(1-methyl-4-piperidinyl)propanol (154 mg, 0.98 mmol) ) in 5 mL of N,N-dimethylformamide was heated at 125° C. for 5 min. Sodium hydride (60%) (98 mg, 2.45 mmol) was added and the mixture was heated at 125° C. for 1 hour. Additional sodium hydride (98 mg, 2.45 mmol) was added and the mixture was heated at 125° C. for 2 hours. The reaction mixture was cooled to room temperature and poured into saturated sodium bicarbonate and stirred for 1 hour. The precipitate was collected, washed with water and dried in vacuo. The residue was purified by preparative thin layer chromatography, developing with 15% methanol in dichloromethane. Trituation with diethyl ether provided 146 mg of 4-[(2,4-dichloro-5-methoxyphenyl)amino]-6-ethoxy-7-[3-(1-methylpiperidin-4-yl)propoxy]quinoline-3-carbonitrile as an off-white solid, mp 148-151° C.

MS 543.2 (M+H)+.

Analysis for C₂₈H₃₂Cl₂N₄O₃-1.8 H₂O.

Calcd: C, 58.39; H, 6.23; N, 9.73. Found: C, 58.40; H, 6.16; N, 9.64.

### EXAMPLE 13

### 4-[(2,4-Dichloro-5-methoxyphenyl)amino]-6-ethoxy-7-[2-(4-methyl-1-piperazinyl)ethoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol) and 2-(4-methyl-1-piperazinyl)ethanol (141 mg, 0.98 mmol) ) in 5 mL of N,N-dimethylformamide was heated to 100° C. Sodium hydride (60%) (196 mg, 4.9 mmol) was added in portions and the mixture was heated at 125° C. for 3 hours. The reaction mixture was cooled to room temperature and treated with 25 mL of water. The mixture was stirred for 2 hours. The precipitate was collected, washed with water and dried in vacuo. The residue was purified by flash column chromatography, eluting with a gradient of 5% methanol in dichloromethane to 15% methanol in dichloromethane. Trituation with diethyl ether provided 123 mg of 4-[(2,4-dichloro-5-methoxyphenyl)amino]-6-ethoxy-7-[2-(4-methyl-1-piperazinyl)ethoxy]quinoline-3-carbonitrile as an off-white solid, mp 141-143° C.

MS 530.2 (M+H)+.

Analysis for C₂₆H₂₉Cl₂N₅O₃.

Calcd: C, 58.87; H, 5.51; N, 13.20. Found: C, 58.48; H, 5.45; N, 12.95.

### EXAMPLE 14

### 4-[(2,4-Dichloro-5-methoxyphenyl)amino]-6-ethoxy-7-[2-(1-methylpiperidin-4-yl)ethoxy]quinoline-3-carbonitrile

A mixture of 4-[(2,4-dichloro-5-methoxyphenyl)amino]-6-ethoxy-7-fluoro-3-quinolinecarbonitrile (200 mg, 0.49 mmol) and 1-methyl-4-piperidinethanol (140 mg, 0.98 mmol) ) in 5 mL of N,N-dimethylformamide was heated to 100° C. Sodium hydride (60%) (162 mg, 4.05 mmol) was added in portions and the mixture was heated at 125° C. for 3 hours. The reaction mixture was cooled to room temperature and treated with 25 mL of water. The precipitate was collected, washed with water and dried in vacuo. The residue was purified by flash column chromatography, eluting with first dichloromethane then a gradient of 5% methanol in dichloromethane to 30% methanol in dichloromethane. Trituation with diethyl ether provided 121 mg of 4-[(2,4-dichloro-5-methoxyphenyl)amino]-6-ethoxy-7-[2-(1-methylpiperidin-4-yl)ethoxy]quinoline-3-carbonitrile as an off-white solid, mp 174-176° C.

MS 529.1 (M+H)+.

Analysis for C₂₇H₃₀Cl₂N₄O₃.

Calcd: C, 61.25; H, 5.71; N, 10.58. Found: C, 61.40; H, 5.84; N, 10.35.

### EXAMPLE 15

### 4-[(2,4-Dichloro-5-methoxyphenyl)amino]-6-methoxy-7-[3-(4-propyl-1-piperazinyl)propoxy]-3-quinolinecarbonitrile

Prepared according to the method used for the preparation of Example 1 by the reaction of 7-[3-chloroethoxy]4-[(2,4-dichloro-5-methoxyphenyl)amino]-6-methoxy-3-quinolinecarbonitrile and N-propylpiperazine: mp 97-101° C.; MS (ES) m/z 558.2, 560.2 (M+1).

What is claimed:

1. A method of providing neuroprotection in a patient following a cerebrovascular ischemic event comprising providing a therapeutically effective amount of a compound of the formula 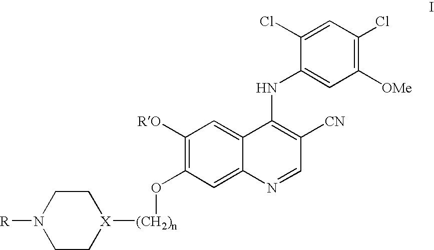
wherein:
X is N, CH
n is an integer from 1-3; and
R′ and R are independently, alkyl of 1 to 3 carbon atoms, and pharmaceutically acceptable salts thereof; with the proviso that when n is 1, X is not N.

2. The method of claim 1 wherein R′ is methyl.

3. The method of claim 1 wherein R is methyl or ethyl.

4. The method of claim 1 wherein X is N.

5. The method of claim 1 wherein X is CH.

6. The method of claim 1 wherein the compound is:
4-[(2,4-Dichloro-5-methoxyphenyl)amino]-6-methoxy-7-[3-(4-methyl-1-piperazinyl)propoxy]-3-quinolinecarbonitrile;
4-[(2,4-Dichloro-5-methoxyphenyl)amino]-7-[3-(4-ethyl-1-piperazinyl)propoxy]-6-methoxy-3-quinolinecarbonitrile;
4-[(2,4-Dichloro-5-methoxyphenyl)amino]-6-methoxy-7-[2-(4-methyl-1-piperazinyl)ethoxy]-3-quinolinecarbonitrile;
4-[(2,4-Dichloro-5-methoxyphenyl)amino]-7-[2-(4-ethyl-1-piperazinyl)ethoxy]-6-methoxy-3-quinolinecarbonitrile;
4-[(2,4-Dichloro-5-methoxyphenyl)amino]-6-methoxy-7-[(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[3-(4-methylpiperazin-1-yl)propoxy]quinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(4-ethylpiperazin-1-yl)propoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile;
4-[(2,4-Dichloro-5-methoxyphenyl)amino]-6-ethoxy-7-[2-(4-methyl-1-piperazinyl)ethoxy]quinoline-3-carbonitrile;
4-[(2,4-Dichloro-5-methoxyphenyl)amino]-6-ethoxy-7-[2-(1-methylpiperidin-4-yl)ethoxy]quinoline-3-carbonitrile; or
4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

7. The method of claim 1 wherein compound is administered between about 6 to about 24 hours after the ischemic event.

8. The method of claim 1 wherein the therapeutically effective amount is from about 1 mg/kg to about 30 mg/kg.

9. The method of claim 1 comprising administering compound of Formula I intravenously.

10. The method of claim 1 wherein the patient is a human.

11. The method of claim 1 wherein the ischemic event is transient.

12. The method of claim 1 wherein the ischemic event is acute.

13. The method of claim 1 wherein the ischemic event is stroke, head trauma, spinal trauma, general anoxia, or hypoxia.

14. The method of claim 1 wherein the ischemic event occurs during cranial hemmorhage, perinatal asphyxia, cardiac arrest or status epilepticus.

15. A method of inhibiting neurological deficits in a patient following a cerebrovascular ischemic event comprising providing a therapeutically effective amount of a compound of the formula 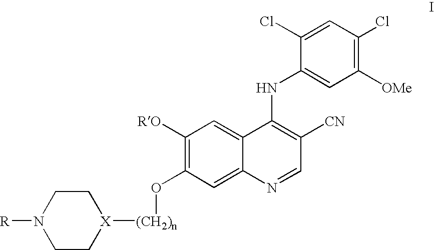
wherein:
X is N, CH
n is an integer from 1-3; and
R′ and R are independently, alkyl of 1 to 3 carbon atoms, and pharmaceutically acceptable salts thereof; with the proviso that when n is 1, X is not N.

16. The method of claim 15 wherein R′ is methyl.

17. The method of claim 15 wherein R is methyl or ethyl.

18. The method of claim 15 wherein X is N.

19. The method of claim 15 wherein X is CH.

20. The method of claim 15 wherein the compound is:
4-[(2,4-Dichloro-5-methoxyphenyl)amino]-6-methoxy-7-[3-(4-methyl-1-piperazinyl)propoxy]-3-quinolinecarbonitrile;
4-[(2,4-Dichloro-5-methoxyphenyl)amino]-7-[3-(4-ethyl-1-piperazinyl)propoxy]-6-methoxy-3-quinolinecarbonitrile;
4-[(2,4-Dichloro-5-methoxyphenyl)amino]-6-methoxy-7-[2-(4-methyl-1-piperazinyl)ethoxy]-3-quinolinecarbonitrile;
4-[(2,4-Dichloro-5-methoxyphenyl)amino]-7-[2-(4-ethyl-1-piperazinyl)ethoxy]-6-methoxy-3-quinolinecarbonitrile;
4-[(2,4-Dichloro-5-methoxyphenyl)amino]-6-methoxy-7-[(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[3-(4-methylpiperazin-1-yl)propoxy]quinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(4-ethylpiperazin-1-yl)propoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile;
4-[(2,4-Dichloro-5-methoxyphenyl)amino]-6-ethoxy-7-[2-(4-methyl-1-piperazinyl)ethoxy]quinoline-3-carbonitrile;
4-[(2,4-Dichloro-5-methoxyphenyl)amino]-6-ethoxy-7-[2-(1-methylpiperidin-4-yl)ethoxy]quinoline-3-carbonitrile; or
4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

21. The method of claim 15 wherein compound is administered between about 6 to about 24 hours after the ischemic event.

22. The method of claim 15 wherein the therapeutically effective amount is from about 1 mg/kg to about 30 mg/kg.

23. The method of claim 15 comprising administering compound of Formula I intravenously.

24. The method of claim 15 wherein the patient is a human.

25. The method of claim 15 wherein the ischemic event is transient.

26. The method of claim 15 wherein the ischemic event is acute.

27. The method of claim 15 wherein the ischemic event is stroke, head trauma, spinal trauma, general anoxia, or hypoxia.

28. The method of claim 15 wherein the ischemic event occurs during cranial hemmorhage, perinatal asphyxia, cardiac arrest or status epilepticus.

29. A method of reducing infarct volumes in a patient following a cerebrovascular ischemic event comprising administering a therapeutically effective amount of a compound of the formula 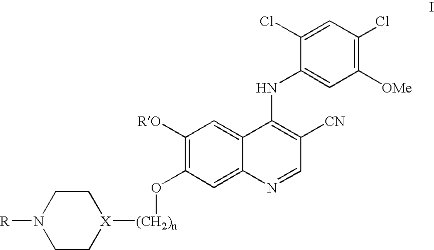
wherein:
X is N, CH
n is an integer from 1-3; and
R′ and R are independently, alkyl of 1 to 3 carbon atoms, and pharmaceutically acceptable salts thereof; with the proviso that when n is 1, X is not N.

30. The method of claim 29 wherein R′ is methyl.

31. The method of claim 29 wherein R is methyl or ethyl.

32. The method of claim 29 wherein X is N.

33. The method of claim 29 wherein X is CH.

34. The method of claim 29 wherein the compound is:
4-[(2,4-Dichloro-5-methoxyphenyl)amino]-6-methoxy-7-[3-(4-methyl-1-piperazinyl)propoxy]-3-quinolinecarbonitrile;
4-[(2,4-Dichloro-5-methoxyphenyl)amino]-7-[3-(4-ethyl-1-piperazinyl)propoxy]-6-methoxy-3-quinolinecarbonitrile;
4-[(2,4-Dichloro-5-methoxyphenyl)amino]-6-methoxy-7-[2-(4-methyl-1-piperazinyl)ethoxy]-3-quinolinecarbonitrile;
4-[(2,4-Dichloro-5-methoxyphenyl)amino]-7-[2-(4-ethyl-1-piperazinyl)ethoxy]-6-methoxy-3-quinolinecarbonitrile;
4-[(2,4-Dichloro-5-methoxyphenyl)amino]-6-methoxy-7-[(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[3-(4-methylpiperazin-1-yl)propoxy]quinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(4-ethylpiperazin-1-yl)propoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile;
4-[(2,4-Dichloro-5-methoxyphenyl)amino]-6-ethoxy-7-[2-(4-methyl-1-piperazinyl)ethoxy]quinoline-3-carbonitrile;
4-[(2,4-Dichloro-5-methoxyphenyl)amino]-6-ethoxy-7-[2-(1-methylpiperidin-4-yl)ethoxy]quinoline-3-carbonitrile; or
4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

35. The method of claim 29 wherein compound is administered between about 6 to about 24 hours after the ischemic event.

36. The method of claim 29 wherein the therapeutically effective amount is from about 1 mg/kg to about 30 mg/kg.

37. The method of claim 29 comprising administering compound of Formula I intravenously.

38. The method of claim 29 wherein the patient is a human.

39. The method of claim 29 wherein the ischemic event is transient.

40. The method of claim 29 wherein the ischemic event is acute.

41. The method of claim 29 wherein the ischemic event is stroke, head trauma, spinal trauma, general anoxia, or hypoxia.

42. The method of claim 29 wherein the ischemic event occurs during cranial hemmorhage, perinatal asphyxia, cardiac arrest or status epilepticus.

43. A method of inhibiting post-ischemic vascular permeability of cerebral blood vessels in a patient suffering from a cerebrovascular event comprising administering a therapeutically effective amount of a compound of the formula 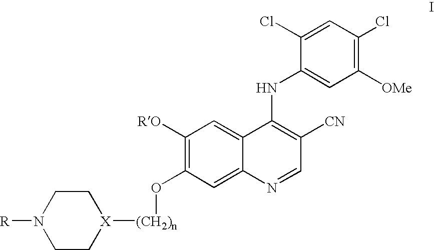
wherein:
X is N, CH
n is an integer from 1-3; and
R′ and R are independently, alkyl of 1 to 3 carbon atoms, and pharmaceutically acceptable salts thereof; with the proviso that when n is 1, X is not N.

44. The method of claim 43 wherein R′ is methyl.

45. The method of claim 43 wherein R is methyl or ethyl.

46. The method of claim 43 wherein X is N.

47. The method of claim 43 wherein X is CH.

48. The method of claim 43 wherein the compound is:
4-[(2,4-Dichloro-5-methoxyphenyl)amino]-6-methoxy-7-[3-(4-methyl-1-piperazinyl)propoxy]-3-quinolinecarbonitrile;
4-[(2,4-Dichloro-5-methoxyphenyl)amino]-7-[3-(4-ethyl-1-piperazinyl)propoxy]-6-methoxy-3-quinolinecarbonitrile;
4-[(2,4-Dichloro-5-methoxyphenyl)amino]-6-methoxy-7-[2-(4-methyl-1-piperazinyl)ethoxy]-3-quinolinecarbonitrile;
4-[(2,4-Dichloro-5-methoxyphenyl)amino]-7-[2-(4-ethyl-1-piperazinyl)ethoxy]-6-methoxy-3-quinolinecarbonitrile;
4-[(2,4-Dichloro-5-methoxyphenyl)amino]-6-methoxy-7-[(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[3-(4-methylpiperazin-1-yl)propoxy]quinoline-3-carbonitrile;
4-[(2,4-Dichloro-5-methoxyphenyl)amino]-6-ethoxy-7-[(1-methylpiperidin-4-yl)methoxy]quinoline-3-carbonitrile; 4-[(2,4-Dichloro-5-methoxyphenyl)amino]-6-ethoxy-7-[3-(4-ethylpiperazin-1-yl)propoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile;
4-[(2,4-Dichloro-5-methoxyphenyl)amino]-6-ethoxy-7-[2-(4-methyl-1-piperazinyl)ethoxy]quinoline-3-carbonitrile;
4-[(2,4-Dichloro-5-methoxyphenyl)amino]-6-ethoxy-7-[2-(1-methylpiperidin-4-yl)ethoxy]quinoline-3-carbonitrile; or
4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

49. The method of claim 43 wherein compound is administered between about 6 to about 24 hours after the ischemic event.

50. The method of claim 43 wherein the therapeutically effective amount is from about 1 mg/kg to about 30 mg/kg.

51. The method of claim 43 comprising administering compound of Formula I intravenously.

52. The method of claim 43 wherein the patient is a human.

53. The method of claim 43 wherein the ischemic event is transient.

54. The method of claim 43 wherein the ischemic event is acute.

55. The method of claim 43 wherein the ischemic event is stroke, head trauma, spinal trauma, general anoxia, or hypoxia.

56. The method of claim 43 wherein the ischemic event occurs during cranial hemmorhage, perinatal asphyxia, cardiac arrest or status epilepticus.

57. A compound having the structure: 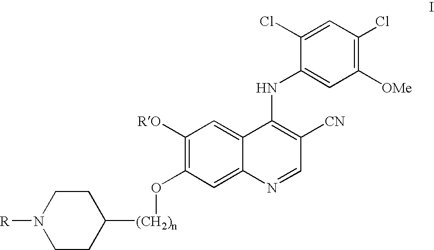
wherein:
n is an integer from 1-3; and
R′ and R are independently, alkyl of 1 to 3 carbon atoms, and pharmaceutically acceptable salts thereof.

58. A compound of claim 57 wherein R′ is methyl.

59. A compound of claim 57 wherein R is methyl or ethyl.

60. A compound which is:
4-[(2,4-Dichloro-5-methoxyphenyl)amino]-6-methoxy-7-(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile; or
4-[(2,4-Dichloro-5-methoxyphenyl)amino]-6-ethoxy-7-[2-(1-methylpiperidin-4-yl)ethoxy]quinoline-3-carbonitrile; and pharmaceutically acceptable salts thereof.

61. A pharmaceutical composition comprising a compound having the structure 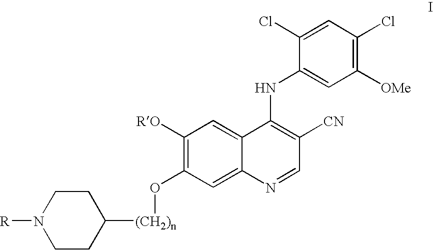
wherein:
n is an integer from 1-3; and
R′ and R are independently, alkyl of 1 to 3 carbon atoms, and pharmaceutically acceptable salts thereof; and a pharmaceutically acceptable carrier or excipient.

62. A pharmaceutical composition of claim 61 comprising a compound which is:
4-[(2,4-Dichloro-5-methoxyphenyl)amino]-6-methoxy-7-(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile; or
4-[(2,4-Dichloro-5-methoxyphenyl)amino]-6-ethoxy-7-[2-(1-methylpiperidin-4-yl)ethoxy]quinoline-3-carbonitrile; and pharmaceutically acceptable salts thereof.

63. A pharmaceutical composition comprising a vascular permeability inhibiting amount of a compound having the structure: 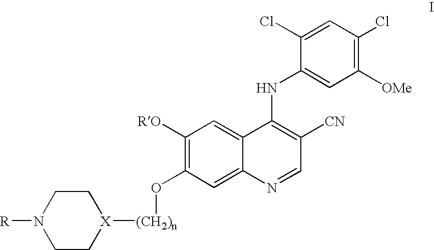
wherein:
X is N, CH
n is an integer from 1-3; and
R′ and R are independently, alkyl of 1 to 3 carbon atoms, and pharmaceutically acceptable salts thereof, with the proviso that when n is 1, X is not N, and a pharmaceutical carrier or excipient.

64. A pharmaceutical composition of claim 63 comprising a compound which is:
4-[(2,4-Dichloro-5-methoxyphenyl)amino]-6-methoxy-7-[3-(4-methyl-1-piperazinyl)propoxy]-3-quinolinecarbonitrile;
4-[(2,4-Dichloro-5-methoxyphenyl)amino]-7-[3-(4-ethyl-1-piperazinyl)propoxy]-6-methoxy-3-quinolinecarbonitrile;
4-[(2,4-Dichloro-5-methoxyphenyl)amino]-6-methoxy-7-[2-(4-methyl-1-piperazinyl)ethoxy]-3-quinolinecarbonitrile;
4-[(2,4-Dichloro-5-methoxyphenyl)amino]-7-[2-(4-ethyl-1-piperazinyl)ethoxy]-6-methoxy-3-quinolinecarbonitrile;
4-[(2,4-Dichloro-5-methoxyphenyl)amino]-6-methoxy-7-[(1-methylpiperidin-4-yl)methoxy]-3-quinolinecarbonitrile;
4-[(2,4-Dichloro-5-methoxyphenyl)amino]-6-methoxy-7-[2-(1-methylpiperidin-4-yl)ethoxy]-3-quinolinecarbonitrile;
4-[(2,4-Dichloro-5-methoxyphenyl)amino]-6-methoxy-7-[3-(1-methylpiperidin-4-yl)propoxy]quinoline-3-carbonitrile;
4-[(2,4-Dichloro-5-methoxyphenyl)amino]-7-[(1-ethylpiperidin-4-yl)methoxy]-6-methoxyquinoline-3-carbonitrile;
4-[(2,4-Dichloro-5-methoxyphenyl)amino]-6-ethoxy-7-[3-(4-methylpiperazin-1-yl)propoxy]quinoline-3-carbonitrile;
4-[(2,4-Dichloro-5-methoxyphenyl)amino]-6-ethoxy-7-[(1-methylpiperidin-4-yl)methoxy]quinoline-3-carbonitrile;
4-[(2,4-Dichloro-5-methoxyphenyl)amino]-6-ethoxy-7-[3-(4-ethylpiperazin-1-yl)propoxy]quinoline-3-carbonitrile;
4-[(2,4-Dichloro-5-methoxyphenyl)amino]-6-ethoxy-7-[3-(1-methylpiperidin-4-yl)propoxy]quinoline-3-carbonitrile;
4-[(2,4-Dichloro-5-methoxyphenyl)amino]-6-ethoxy-7-[2-(4-methyl-1-piperazinyl)ethoxy]quinoline-3-carbonitrile;
4-[(2,4-Dichloro-5-methoxyphenyl)amino]-6-ethoxy-7-[2-(1-methylpiperidin-4-yl)ethoxy]quinoline-3-carbonitrile; or
4-[(2,4-Dichloro-5-methoxyphenyl)amino]-6-methoxy-7-[3-(4-propyl-1-piperazinyl)propoxy]-3-quinolinecarbonitrile; and pharmaceutically acceptable salts thereof.

65. The composition of claim 63 in an intravenous dosage form.
